(19) United States
(12) Patent Application Publication
(10) Pub. No.: US 2005/0211242 A9
(43) Pub. Date: Sep. 29, 2005

(54) An apparatus for producing a catalytic reaction

(76) Inventor: Peter Jorg Plath, Langwedel (DE)

Correspondence Address: MERCHANT & GOULD PC, P.O. BOX 2903, MINNEAPOLIS, MN, 55402-0903, US

(21) Appl. No.: 10/332,110

(22) PCT Filed: Jul. 4, 2001
(86) PCT No.: PCT/DE01/02509

§ 371 Date: Aug. 25, 2003

(30) Foreign Application Priority Data
Jul. 5, 2000 (DE) 10032059.7

(51) Int. Cl. A61M 15/00

(52) U.S. Cl. 128/203.12

(57) ABSTRACT

The invention relates to an apparatus for bringing about a catalytic pipe reaction. The apparatus comprises an arrangement of a plurality of microreactors with a respective channel each being formed between adjacent microreactors in such a manner that the catalytic pipe reaction can become effective along the arrangement of the plurality of microreactors.

The invention is concerned with an apparatus for bringing about a catalytic pipe reaction and relates more specifically to a catalytic converter means for motor vehicles.

Apparatus of the kind in question are reaction devices in which a chemical pipe reaction propagates through the reaction devices or develops along the same. A pipe reaction is made use of, for instance, for the preparation of sulfuric acid or for high density ethylene polymerization or continuous styrene polymerization.

It is known to have a catalyst conveniently disposed at the path along which the pipe reaction through the reaction apparatus takes place so that the pipe reaction will turn out to be optimal. Such a catalyst may be distributed uniformly along the path. Also, it may be necessary to have different concentrations of the catalyst present in the different portions of the reaction apparatus. To this end, a catalytically inactive mixture is added to the catalyst. The ratio between the catalyst and the admixture which particularly serves to dilute the catalyst varies among the various sections along the reaction apparatus.

It is the object of the instant invention to provide an apparatus of the kind defined initially with improved distribution of the catalyst along the reaction device formed by the apparatus.

This object is met, in accordance with the invention, with an apparatus as defined in the preamble of claim 1, by an arrangement composed of a plurality of microreactors, with channels being formed each between adjacent microreactorsÿso as to produce the catalytic pipe reaction along the arrangement of the plurality of microreactors.

The essential advantage achieved by the invention over the state of the art is obtained by the possibility of providing a respective predetermined, preferably very small amount of catalyst for the pipe reaction in the plurality of microreactors along the way. In this manner, catalyst can be made available in an amount depending on the particular state of the pipe reaction in the respective microreactor to assure optimum continuance of the reaction.

According to a convenient further development of the invention substance volume transport is established between adjacent microreactors whereby the adjacent microreactors are coupled in terms of substance volume. This means that reactions requiring transportation of intermediate reaction products can be produced in the apparatus.

It is convenient to have thermal transport through the respective channel between adjacent microreactors so that the adjacent microreactors will be coupled thermally. Hereby, heat transmission is obtained between the adjacent microreactors.

In a further development of the invention, a respective channel is designed to have a length which will prevent reverse diffusion of the substance volume from one of the adjacent microreactors into another one connected upstream thereof in the direction of propagation of the reaction. That prevents the partial reaction of the pipe reaction under way in the other one of the adjacent microreactors from retroactively influencing the partial reaction of the pipe reaction taking place in the one of the adjacent microreactors.

It may be an advantage to provide for one or all of the plurality of microreactors to comprise means for influencing at least one reaction parameter of the catalytic reaction in the respective microreactor as that permits purposively varying reaction parameters of individual sections along the way of the pipe reaction as it passes through the apparatus.

According to a preferred embodiment of the invention, means for influencing at least one reaction parameter of the catalytic reaction in a first one of the plurality of microreactors are coupled to sensor means for detecting at least one reaction parameter of the catalytic reaction in a second one of the plurality of microreactors. Hereby, the coupling of the catalytic reaction in the first one of the plurality of microreactors with the catalytic reaction in the second one of the plurality of microreactors can be devised such that the at least one reaction parameter of the catalytic reaction in the first one of the plurality of microreactors can be influenced in response to the at least one reaction parameter of the catalytic reaction in the second one of the plurality of microreactors, as detected by the sensor means. In this manner the catalytic reaction can be optimized by specifically influencing parts of the ongoing reaction in the plurality of microreactors.

In a preferred further development of the invention it may be provided that the means for influencing the at least one reaction parameter of the catalytic reaction in the second one of the plurality of microreactors are coupled to sensor means for detecting at least one reaction parameterÿof the catalytic reaction in the first one of the plurality of microreactors so that coupling of the catalytic reaction in the second one of the plurality of microreactors with the catalytic reaction in the first one of the plurality of microreactors can be obtained in such a way that influencing of the at least one reaction parameter of the catalytic reaction in the second one of the plurality of microreactors can be achieved in response to the at least one reaction parameter of the catalytic reaction in the first one of the plurality of microreactors, as detected by the sensor means. This permits mutual influencing of the sections of the pipe reaction effective in the first and second of the plurality of microreactors.

In a convenient modification of the invention it is provided that the means for influencing at least one of the reaction parameters in the respective microreactor comprise a control means for temperature control in the respective microreactor. Hereby, the temperature may be raised or lowered so as to yield optimum pipe reaction.

The means for influencing at least one of the reaction parameters in the respective microreactor advantageously comprise an irradiation means for radiating light into the respective microreactor. This offers a possibility of either accelerating or protracting a photosensitive pipe reaction.

In a further development of the invention it is provided that the means for influencing at least one reaction parameter in the respective microreactor comprise supply means for introducing at least one additional reactive substance (educt) into the respective microreactor. The introduction of additional reactive substances may either accelerate or protract the pipe reaction. It is likewise possible to introduce inert substances that are required to sustain the pipe reaction, such as a carrier gas or a solvent.

It is advantageous if a state of the respective microreactor, in particular a fixed point state, a bistable state, an oscillator state, or a state of chaos is adjustable by the means for influencing at least one reaction parameter in the respective microreactor. In this manner the respective most favorable state in the respective microreactor is selectable for supporting the pipe reaction in a particular portion of the pipe reaction.

In a convenient modification of the invention, adjacent microreactors are disposed so as to be laterally offset with respect to each other. The offset, for instance, may be from about 0.05 mm to about 30 mm. That reduces the space requirement of the apparatus.

To the same end it may be provided, advantageously, that opposed sidewalls of the plurality of microreactors have a spacing between them of from about 0.02 mm to about 5 mm.

The reaction apparatus for a pipe reaction, obtained by sequentially arranging a plurality of microreactors, may be utilized benefitially as a catalytic converter means for motor vehicles. With this application, the structural design of the individual microreactors should be such that there is sufficiently great probability that the exhaust gas particles in the microreactor will contact a respective catalyst assembly, preferably made of palladium or platinum.

The invention will be described further, by way of example, with reference to the accompanying drawings, in which:

FIG. 1 is a diagrammatic illustration of an apparatus for producing a pipe reaction;

FIG. 2 is a cross sectional view of the apparatus shown in FIG. 1;

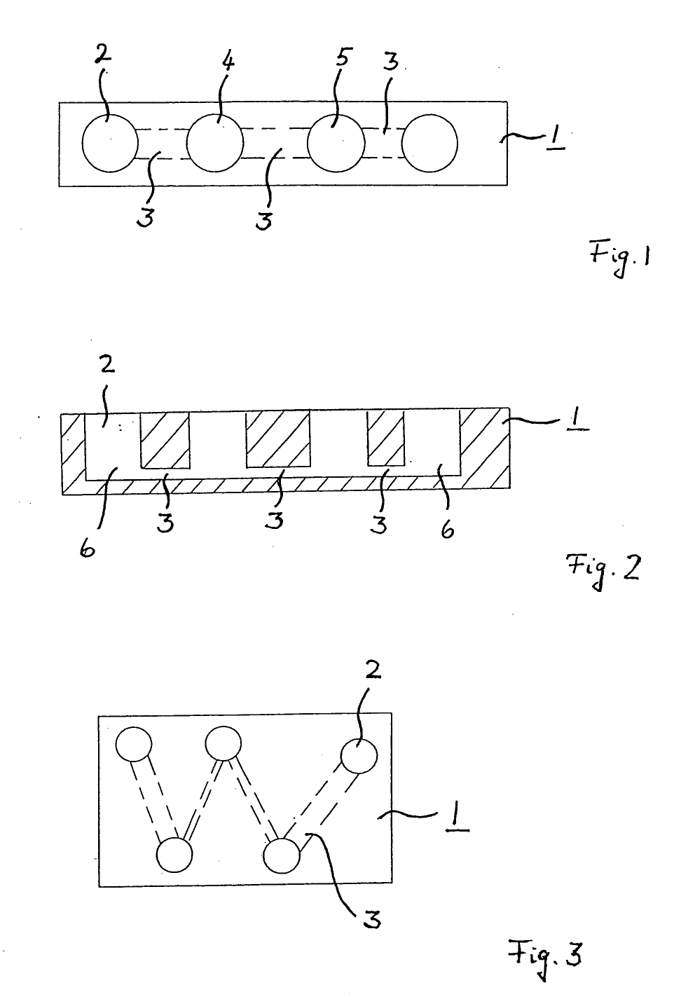

FIG. 3 illustrates another embodiment of an apparatus for producing a pipe reaction;

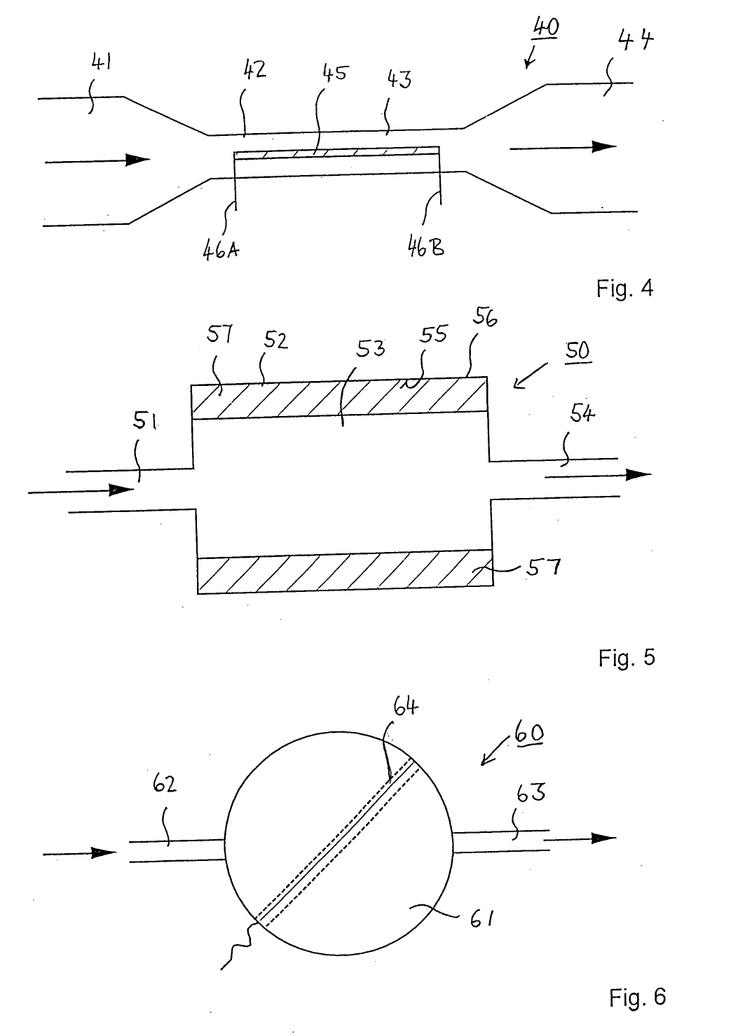

FIG. 4 is a diagrammatic cross sectional view of a microreactor, especially for a motor vehicle catalytic converter;

FIG. 5 is a diagrammatic cross sectional view of another microreactor, especially for a motor vehicle catalytic converter;

FIG. 6 is a diagrammatic top plan view of another microreactor, especially for a motor vehicle catalytic converter;

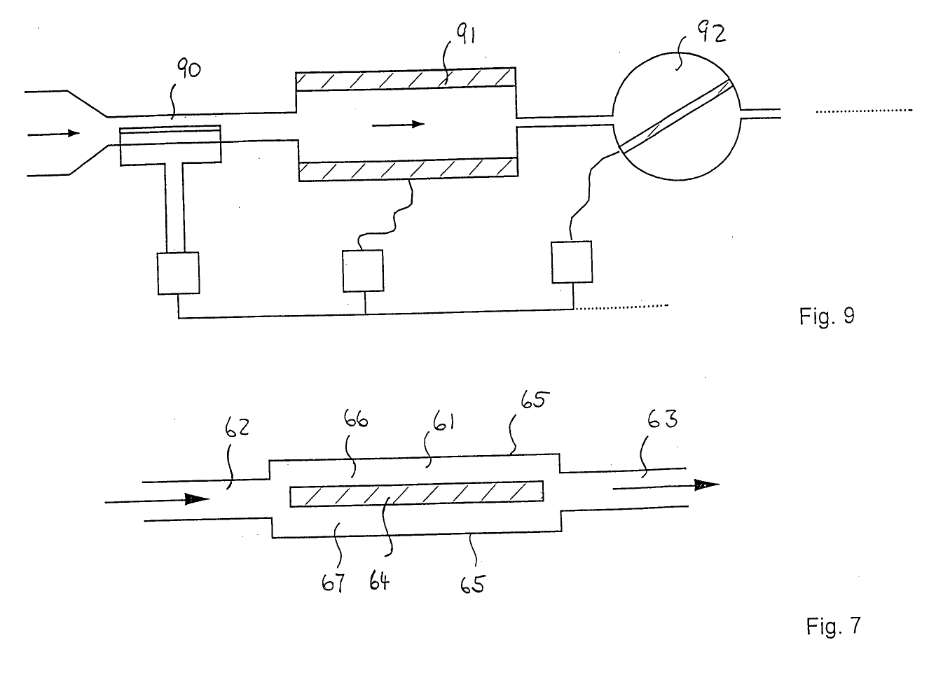

FIG. 7 is a cross sectional view of the microreactor shown in FIG. 6;

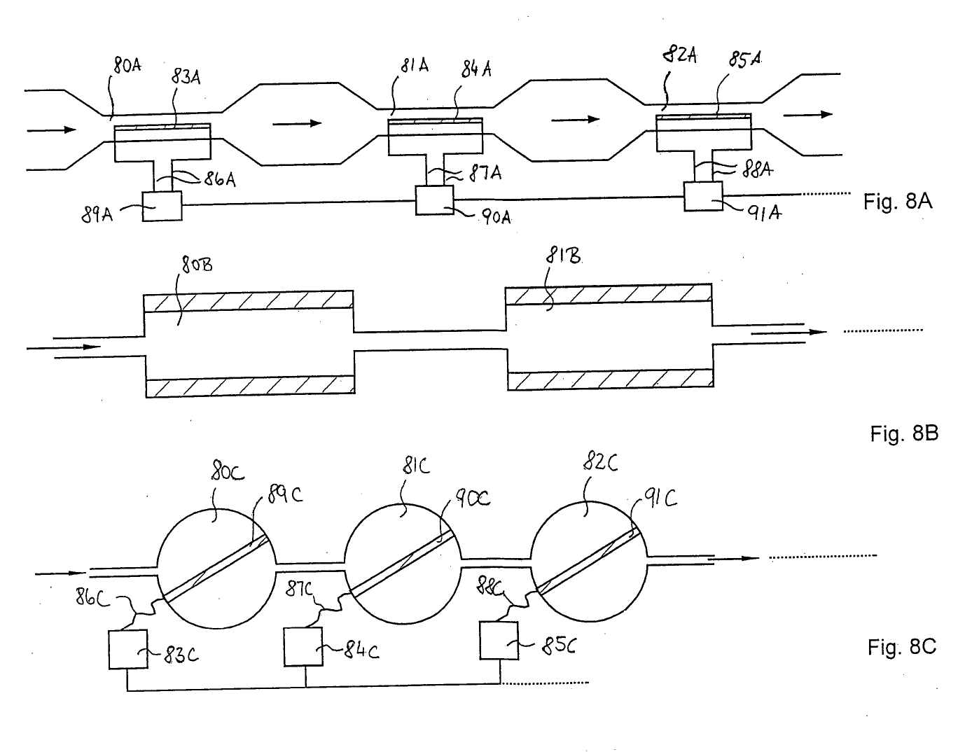

FIG. 8A to 8C show catalyst means, each comprising a combination of like microreactors as shown in FIGS. 4, 5, or 6;

FIG. 9 shows a catalyst means comprising a combination of different microreactors; and

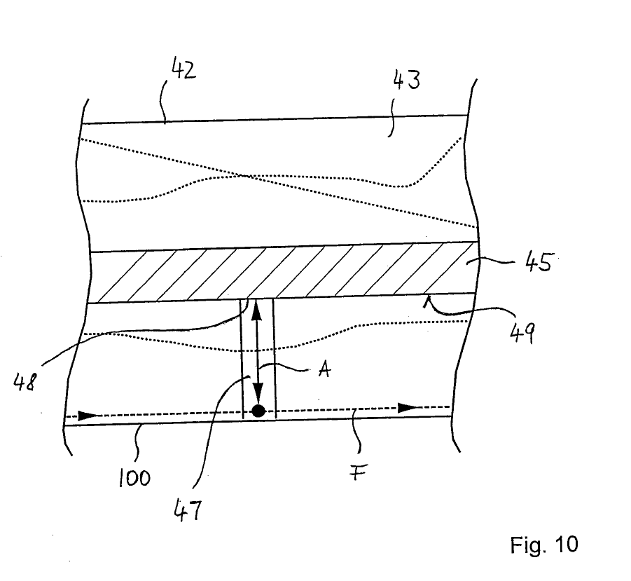

FIG. 10 is a diagrammatic cross sectional view of part of the microreactor shown in FIG. 4, on an enlarged scale.

As may be seen in FIG. 1, an apparatus 1 for producing a catalytic pipe reaction comprises a plurality of sequentially arranged microreactors 2. Adjacents ones of the plurality of microreactors 2 are interconnected by a channel 3 each. The apparatus 1 represents a novel microreactor or micro pipe reactor which comprises the plurality of microreactors 2.

When a catalytic pipe reaction is under way in the apparatus 1 the plurality of microreactors 2 and the respective channels 3 between adjacents one of them form an integral reaction device. The ongoing catalytic pipe reaction is carried on between adjacent microreactors by substance volume transport and/or thermal transport which take place between the adjacent microreactors in the direction of propagation of the pipe reaction. Provision may be made for such thermal transport between the plurality of microreactors 2 to take place also in a direction opposed to the propagating pipe reaction. That may serve for directly influencing the pipe reaction in a first one of the plurality of microreactors 2 in response to the course or state of the pipe reaction in a second one of the plurality of microreactors 2, the second of the plural microreactors 2 being connected upstream of the first of the plural microreactors 2 in the direction of the reaction. The first and second of the plurality of microreactors 2 may be adjacent ones. The thermal transport between the first and second of the plurality of microreactors 2 may be accomplished by a thermal bridge (not shown). The thermal bridge, for instance, may comprise a temperature sensor at the first of the plurality of microreactors 2, a heater/cooler element at the second of the plurality of microreactors 2, and an electrical coupling between the temperature sensor and the heater/cooler element. This would be an electrical thermal bridge. It is likewise possible to use thermal bridges which essentially rely on heat conduction. The thermal bridge may be utilized for influencing the reaction either in the direction of the propagating pipe reaction or in the opposite direction.

As regards their shape, length, width, and height, the channels 3 are designed such that reverse diffusion of the substance volume is impeded between any two adjacent ones of the plurality of microreactors 2 so that it is warranted that the pipe reaction will propagate in one direction. The channels 3 may all be of the same design. But some channels 3 may also be designed differently from others.

The sequential arrangement of the microreactors 2 which are interconnected by the channels 3, on the one hand, provides an integral reaction apparatus for producing the pipe reaction. On the other hand, part of the pipe reaction in one of the plurality of microreactors 4 also may be influenced irrespective of another part of the pipe reaction which is ongoing in another one of the plurality of microreactors 5.

FIG. 2 is a cross sectional view of the apparatus shown in FIG. 1. The channels 3 link the plurality of microreactors 2 in a lower region 6 each of the plurality of microreactors 2.

One or each one of the plurality of microreactors 2 may be equipped with means (not shown) for purposively varying reaction parameters or reaction conditions in the respective microreactor in order thus to initiate, accelerate, or impede the pipe reaction in the integral reaction apparatus which is composed of the plurality of microreactors 2 and channels 3. The reaction conditions in a certain microreactor are influenced by the means provided for that particular microreactor. Coupling or joint control may be provided for the means associated with microreactors. The overall course of the catalytic pipe reaction may be influenced by changing the reaction parameters or the reaction conditions in one of the microreactors.

The means for purposively changing the reaction parameters or reaction conditions, in particular, may comprise a control means for temperature control in the respective microreactor and/or an irradiation means for radiating light into the respective microreactor. The means by which to vary the reaction parameters or the reaction conditions may be utilized for separately creating reaction conditions in the plurality of microreactors so as to exert the desired influence on the pipe reaction. Thus a state of a respective microreactor may be adjusted so that this particular microreactor will be in a fixed point state, an oscillator state, or a state of chaos.

It may be advantageous in connection with the various means for purposively varying the reaction parameters or reaction conditions in single microreactors or multiple microreactors 2 to provide for this targeted change in one or more microreactors of the apparatus 1 to be made in response to the state or course of the pipe reaction in one or more other microreactors of the apparatus 1.

Furthermore, provision may be made in one or all of the plurality of microreactors 2 for additional reactive or inert substances and/or catalysts to be introduced into the respective microreactor so as to influence the part of the pipe reaction taking place in that particular microreactor or the whole pipe reaction. To allow that, the microreactors for which such influencing is desired are provided with supply means, such as openings in the microreactors, connections, etc. The additional substances or catalysts may be introduced, for instance, by means of micropumps.

FIG. 3 diagrammatically shows another embodiment of the apparatus 1 for producing a pipe reaction. The plurality of microreactors 2 are offset laterally with respect to each other. Adjacent ones of the plurality of microreactors are interconnected through channels 3. The arrangement shown in FIG. 3 of the plurality of microreactors 2 and the respective channels 3 allows building the apparatus 1 as a compact space saving structure.

FIGS. 4 to 7 illustrate microreactors intended for use in catalytic converter means of motor vehicles which are run on petroleum based fuel. Several of the microreactors shown in FIGS. 4 to 6 are connected in series to form a catalytic converter means for motor vehicles. A micro pipe reactor thus is formed of the microreactors wherein at least substance volume flow takes place during the catalytic reaction. The desired catalytic effect on the reaction products of the fuel conversion may be obtained by a combination exclusively of microreactors of the same kind, i.e. according to either FIGS. 4, 5, or 6 alone (cf. FIGS. 8A to 8C) or of microreactors of different types, in other words, for example, one microreactor according to FIG. 4, one microreactor according to FIG. 5, and one microreactor according to FIG. 6 (cf. FIG. 9).

FIG. 4 is a cross sectional elevation in a longitudinal direction and shows a microreactor 40 having a supply portion 41, a central portion 42 inside of which a reaction chamber 43 is defined, and an end portion 44. Throughout the range of the supply portion 41, the central portion 42, and the end portion 44, the microreactor 40 preferably has a substantially round cross section transversely of the longitudinal direction. The reaction chamber 43 houses a catalyst assembly 45 around which gas particles can flow and which comprises electrical connections 46A, 46B. The electrical connections 46A, 46B may be used to heat the catalyst assembly 45 for control and/or optimization of the catalytic reaction of the (exhaust) gas particles which enter the reaction chamber 43. In the case of catalytic converters for motor vehicles the catalyst assembly 45 typically is made of platinum or palladium. Other materials may be used as well, as long as they can produce sufficient catalytic reaction. The catalyst assembly 45 preferably is embodied by a wire or wire mesh or wire network. The selection of a suitable kind of catalyst assembly 45 depends on the particular application. In shape/dimension, the catalyst assembly 45 and the reaction chamber 43 must be designed in consideration of the fact that, inside the reaction chamber 43, a gas particle, in moving through the reaction chamber 43 to get from the supply portion 41 to the end portion 44, must pass at least once with a sufficiently high degree of probability through a reaction portion 47 of the reaction chamber 43, at a distance A between the gas particle and a portion 48 (cf. FIG. 10) on an outer surface 49 of the catalyst assembly 45, as seen in a direction perpendicular to the tangent to a trajectory F of the gas particle in the reaction portion 47, preferably smaller than or equal to ten times the mean transverse diffusion length of the gas particle. In this manner it can be assured that the gas particle will impinge at least once on the surface of the catalyst assembly 45. In the case of microreactor 40 according to FIG. 4, with the worst case scenario, the gas particles could move in very close proximity to a wall 100 of the central portion 42, as indicated in FIG. 10 by trajectory F. Other possible paths of movement are indicated in dotted lines in the same figure. Looking at the embodiment illustrated in FIG. 4 and FIG. 10, it follows essentially from the design requirement described that at least in a partial area of the reaction chamber 43 a distance between the wall 100 and the surface 49 of the catalyst assembly 45 must be smaller than or equal to a small-number multiple of the transverse diffusion length of the gas particle, and that there must be a sufficiently high degree of probability that the gas particles enter that partial area of the reaction chamber 43.

The catalyst assembly 45 and the reaction chamber 43 or the central portion 42 must be so designed that an interaction most likely will take place due to the relative relationship between the distance A and the small-number multiple of the mean transverse diffusion length of the gas particle, to initiate a catalytic reaction of the gas particle, including the material of the catalyst assembly 45. In this manner a high catalytic efficiency of the microreactor 40 is achieved. Those skilled in the art, while observing the design characteristics specified above, can choose different structures in which a desired catalytic effect is obtained.

Conveniently, the design requirements described above are to be applied so that a gas particle passing through the microreactor will enter into an effective reaction space, and the dwelling time within the reaction space will be so long that there is sufficient likelihood that a catalytic reaction of the gas particle, including the respective catalyst assembly, will occur.

FIGS. 5, 6 and 7 show other embodiments of microreactors 50 and 60, respectively, suitable to be designed according to the characteristics mentioned above and to be used in catalytic converters for motor vehicles. The microreactor 50 shown in cross section along a longitudinal direction comprises a supply portion 51 through which reactants enter into a central portion 52 which includes a reaction chamber 53. Having passed the reaction chamber 53, the gas particles enter an end portion 54 of the microreactor 50. Inside the reaction chamber 53, a catalyst assembly 57 is fixed on an inner surface 55 of a wall 56 in the central portion 52. This catalyst assembly may be embodied by a surface coating which covers the inner surface 55 totally or in part. Suitable materials for the catalyst assembly 57, for example, are platinum or palladium. As regards the structural features of the reaction chamber 53 and the catalyst assembly 57, particularly the dimensioning and external configuration of the catalyst assembly 57, the requirements set out above, for microreactor 40, in respect of a partial area in the reaction chamber through which the gas particles must pass, apply accordingly. It follows from the design requirement specified above that, in a microreactor 50 having, for example, a round cross section transversely of the longitudinal direction in the range of the central portion 52 and an internal surface 57 of the central portion 52 covered substantially fully by catalyst material, that the radius of the round cross section transversely of the longitudinal direction in the area defined by the catalyst assembly 57 must be smaller than or equal a small-number multiple of the mean transverse diffusion length of the gas particles.

FIGS. 6 and 7 are two representations of another microreactor 60 comprising a sandwich-like reaction chamber 61 (FIG. 7) which the gas particles enter through a supply portion 62 and, having passed it, leave through an end portion 63. The reaction chamber 61 houses a catalyst assembly 64, preferably of wire or net-like structure, around which the gas particles flow. The catalyst assembly 64 and a wall 65 in the area of the reaction chamber 61 are designed in such a way that the requirements specified in connection with the microreactor 40 regarding the dimension and the movement at least once past a reactor portion (reference 46 in FIG. 4) are fulfilled in an area 66 above the catalyst assembly 64 and/or an area 67 below the catalyst assembly 64. For this reason there is sufficient likelihood of obtaining catalytic reaction of the gas particles which enter the reaction chamber 61.

Apart from the design feasibilities illustrated in FIGS. 4, 5, and 6 or 7 for microreactors 40, 50, and 60, a person skilled in the art, while observing the structural features specified above for the area of the respective reaction chamber in which the catalyst assembly is positioned, may devise other embodiments of microreactors, exploiting the improved catalytic action obtained when the structural requirements set forth are met.

A further increase in efficiency of the catalytic action is obtained by ligning up a plurality of microreactors 40, 50, or 60, whereby a reaction apparatus is formed in which substance volume transport takes place between the microreactors connected in series when a catalytic pipe reaction is under way. Possible embodiments of such an apparatus comprising a plurality of microreactors 80A, 81A, 82A; 80B, 81B; 80C, 81C, 82C connected in series are illustrated in FIGS. 8A, 8B, and 8C, respectively. Each one of the microreactors 80A, 81A, 82A; 80B, 81B; 80C, 81C, 82C is designed in accordance with the structural features defined above of microreactors 40, 50, and 60, respectively.

Catalyst assemblies 83A, 84A, 85A of the microreactors 80A, 81A, and 82A, respectively, each are connected by a respective electrical connection 86A, 87A, 88A to a control means 89A, 90A, and 91A, respectively. The control means 89A, 90A, 91A serve to control the supply of current to the catalyst assemblies 83A, 84A, 85A for heating the catalyst assemblies 83A, 84A, 85A in order to optimize the respective catalytic reaction. A superordinated control means (not shown) may be provided to influence the cooperation between the control means 89A, 90A, 91A. In this manner the catalytic reaction taking place along the microreactors 80A, 81A, and 82A, respectively, can be optimized so as to reduce the content of noxious components in the fuel for operating a fuel cell or in the exhaust gas of a motor vehicle when the apparatus including the microreactors 80A, 81A, 82A is employed.

In analogous manner, the microreactor combinations 80B and 81B as well as 80C, 81C, 82C illustrated in FIGS. 8B and 8C, respectively, may be furnished with respective control means (not shown in FIG. 8B) in order to control the reactions produced in the individual microreactors. As shown in FIG. 8C, the microreactor arrangement comprises control means 83C, 84C, 85C connected to catalyst assemblies 89C, 90C, 91C through respective connections 86C, 87C, 88C.

In the embodiments according to FIGS. 8A to 8C the microreactors connected in series all are of the same structure. As FIG. 9 shows, however, it is likewise possible to combine microreactors 90, 91, 92 having different structural features if that is required for a particular application in order to provide the desired catalytic reaction. Any desired number of like or unlike microreactors may be combined to form an apparatus suitable to produce the catalytic pipe reaction, as the particular application may require.

The microreactors or microreactor arrangements described above help prevent the undesirable pressure loss in a motor vehicle catalytic converter means that occurs with conventional catalytic converters for motor vehicles. Moreover, the above mentioned control means, such as control means 83C, 84C, 85C in FIG. 8C, permit temperature control in the individual microreactors so that a desired substantially constant temperature distribution can be achieved along the reaction path of the gas particles and any overheating of individual sections of a catalyst assembly for motor vehicles, as frequently happens with conventional catalytic converters, can be avoided. Thus a method is provided of operating a catalyst means including a plurality of microreactors with which a pipe reaction unfolding along the catalyst means can be controlled and optimized by way of temperature control in the microreactors. The temperature control may be provided either for individual ones or all of the microreactors. If optimizing should be required by way of influencing another reaction parameter than the temperature, suitable control means for such purpose may be selected.

The features of the invention disclosed in the specification above, in the drawing and claims may be essential to implementing the invention in its various embodiments, both individually and in any combination.

1. An apparatus (1) for effecting a catalytic pipe reaction, characterized by an arrangement of a plurality of microreactors (2), respective channels (3) being formed each between adjacent microreactors so as to produce the catalytic reaction along the arrangement of the plurality of microreactors (2).

2. The apparatus (1) as claimed in claim 1, characterized in that substance volume transport between adjacent microreactors is established through the respective channel (3) so that the adjacent microreactors are coupled in terms of substance volume.

3. The apparatus (1) as claimed in claim 1, characterized in that thermal transport between adjacent microreactors is established through the respective channel (3) so that the adjacent microreactors are coupled thermally.

4. The apparatus (1) as claimed in claim 1, characterized in that a length of the respective channel (3) is designed to prevent reverse diffusion of the substance volume from one of the adjacent microreactors (5) into another one of the adjacent microreactors (4) contrary to the direction of propagation of the catalytic pipe reaction.

5. The apparatus (1) as claimed in claim 1, characterized in that one or all of the plurality of microreactors (2) comprise means for influencing at least one reaction parameter of the catalytic reaction in the respective microreactor.

6. The apparatus (1) as claimed in claim 5, characterized in that the means for influencing at least one reaction parameter of the catalytic reaction in a first one of the plurality of microreactors (2) are coupled to sensor means for detecting at least one reaction parameter of the catalytic reaction in a second one of the plurality of microreactors (2) so that coupling of the catalytic reaction in the first one of the plurality of microreactors (2) with the catalytic reaction in the second one of the plurality of microreactors (2) can be obtained in such a way that influencingÿof the at least one reaction parameter of the catalytic reaction in the first one of the plurality of microreactors (2) can be achieved in response to the at least one reaction parameter of the catalytic reaction in the second one of the plurality of microreactors (2) detected by the sensor means.

7. The apparatus (1) as claimed in claim 5, characterized in that the means for influencing at least one reaction parameter of the catalytic reaction in the second one of the plurality of microreactors (2) are coupled to sensor means for detecting at least one reaction parameter of the catalytic reaction in the first one of the plurality of microreactors (2) so that coupling of the catalytic reaction in the second one of the plurality of microreactors (2) with the catalytic reaction in the first one of the plurality of microreactors (2) can be obtained in such a way that influencingÿof the at least one reaction parameter of the catalytic reaction in the second one of the plurality of microreactors (2) can be achieved in response to the at least one reaction parameter of the catalytic reaction in the first one of the plurality of microreactors (2) detected by the sensor means.

8. The apparatus (1) as claimed in claim 5, characterized in that the means for influencing at least one reaction parameter in the respective microreactor comprise a control means for temperature control in the respective microreactor.

9. The apparatus (1) as claimed in claim 5, characterized in that the means for influencing at least one reaction parameter in the respective microreactor comprise an irradiation means for radiating light into the respective microreactor.

10. The apparatus (1) as claimed in claim 5, characterized in that the means for influencing at least one reaction parameter in the respective microreactor comprise supply means for introducing at least one additional reactive or inert substance into the respective microreactor.

11. The apparatus (1) as claimed in claim 5, characterized in that a state of the respective microreactor, especially a fixed point state, a bistable state, an oscillator state, or a state of chaos, is adjustable by the means for influencing at least one reaction parameter in the respective microreactor.

12. The apparatus (1) as claimed in claim 1, characterized in that adjacent microreactors are arranged laterally offset with respect to each other, for example by from about 0.05 mm to about 30 mm.

13. The apparatus (1) as claimed in claim 1, characterized in that opposed sidewalls of the plurality of microreactors (2) are spaced from each other by from about 0.02 mm to about 5 mm.

14. The apparatus as claimed in claim 1, wherein the plurality of microreactors each comprise a catalyst assembly.

15. Use of an apparatus (1) as claimed in claim 1 as a micro pipe reactor.

16. Use of an apparatus as claimed in claim 14 as a catalytic converter means for motor vehicles.

17. An apparatus for effecting a catalytic reaction, especially a catalytic reaction of fuel for fuel cells or of exhaust gas particles of motor vehicles, comprising a supply portion, a central portion in which a reaction chamber is defined, and an end portion, characterized in that a catalyst assembly is arranged in the reaction chamber so that at least once the gas particles in the reaction chamber will enter a reaction portion in which a distance between the gas particles and a portion on an outer surface of the catalyst assembly, in a direction perpendicular to the tangent to a trajectory of the gas particles in the reaction portion, is smaller than or equal to a small-number multiple of the mean transverse diffusion length of the gas particles.

18. The apparatus as claimed in claim 17, characterized in that the distance is smaller than or equals ten times the mean transverse diffusion length of the gas particles.

19. An arrangement comprising a plurality of apparatus as defined in claims 17 or 18 for producing a catalytic pipe reaction.
